(19) United States
(12) Patent Application Publication
(10) Pub. No.: US 2004/0235916 A1
(43) Pub. Date: Nov. 25, 2004

(54) Thiazolyl amides and their use as antiviral drugs

(76) Inventors: Rudolf Schohe-Loop, Wuppertal (DE); Ulrich Betz, Wuppertal (DE); Rudiger Fischer, Pulheim (DE); Martin Hendrix, Odenthal (DE); Gerald Kleymann, Bad Salzuflen (DE); Judith Baumeister, Wuppertal (DE); Wolfgang Bender, Wuppertal (DE); Peter Eckenberg, Wuppertal (DE); Gabriele Handke-Erguden, Wulfrath (DE); Kerstin Henninger, Wuppertal (DE); Axel Jensen, Velbert (DE); Jorg Keldenich, Wuppertal (DE); Udo Schneider, Leverkusen (DE); Olaf Weber, Wulfrath (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/481,672

(22) PCT Filed: Jun. 10, 2002
(86) PCT No.: PCT/EP02/06324

(30) Foreign Application Priority Data
Jun. 22, 2001 (DE) 101 29 715.7

(51) Int. Cl. A61K 31/427; A61K 31/426; C07D 417/02; C07D 277/38

(52) U.S. Cl. 514/369; 548/181; 548/184

(57) ABSTRACT

The invention relates to the novel compounds of formula (I), to a method for producing the same and to their use as drugs, especially as antiviral drugs.

The present invention relates to novel compounds, namely thiazolylamides, to processes for their preparation and to their use as medicaments, in particular as antiviral medicaments.

WO 00/01498 relates to thiazolylureas with anti-herpes virus properties. WO 00/05114 relates to indolinylureas with anti-herpes virus properties. WO 97/24343 and WO 99/42455 relate to phenylthiazole derivatives with anti-herpes virus properties.

The present invention relates to novel compounds which are thiazolylamide derivatives of the general formula (I):

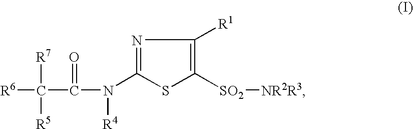

in which

R¹ is hydrogen, halogen, (C₁-C₆)-alkyl, (C₁-C₆)-alkoxy, amino-(C₁-C₆)-alkyl or halo-(C₁-C₆)-alkyl,

R² is hydrogen, (C₁-C₆)-alkoxy, (C₃-C₈)-cycloalkyl or biphenylaminocarbonyl, or

is (C₁-C₆)-alkyl which is optionally substituted by 1 to 3 substituents which are selected from the group consisting of (C₃-C₆)-cycloalkyl, (C₁-C₆)-alkoxy, halogen, hydroxyl, amino, tri-(C₁-C₆)-alkylsilyloxy, radicals of the formula

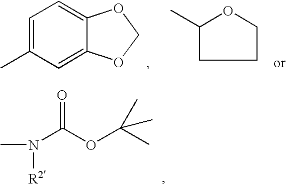

in which R^{2′} is hydrogen or (C₁-C₄)-alkyl,

a 5- to 6-membered aromatic heterocycle having up to 3 heteroatoms from the series S, N and/or O, where a nitrogen-containing heterocycle may also be bonded via the nitrogen atom,

a 3- to 8-membered saturated or unsaturated, nonaromatic, heterocycle which is optionally bonded via a nitrogen atom and has up to 3 heteroatoms from the series S, N and/or O, and

(C₆-C₁₀)-aryl which may in turn be substituted by hydroxyl or (C₁-C₆)-alkoxy, or

is a group of the formula

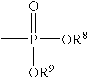

in which R⁸ and R⁹ are identical to or different from one another and are hydrogen and (C₁-C₄)-alkyl, or

are a group of the formula

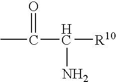

in which R¹⁰ is the side group of a naturally occurring α-amino acid, or

is a group of the formula

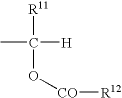

in which R¹¹ is (C₁-C₄)-alkyl, and R¹² is hydrogen, (C₁-C₄)-alkyl or a group of the formula

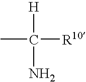

in which R¹¹ is the side group of a naturally occurring α-amino acid,

or in which

R² is (C₃-C₈)-cycloalkyl,

which is optionally substituted by 1 to 3 substituents selected from the group consisting of (C₁-C₆)-alkyl, (C₃-C₆)-cycloalkyl, (C₁-C₆)-alkoxy, trifluoromethyl, trifluoromethoxy, halogen, hydroxyl, amino and nitro,

R³ is (C₃-C₈)-cycloalkyl,

which is substituted by 1 to 3 substituents selected from the group consisting of (C₁-C₆)-alkyl, (C₃-C₆)-cycloalkyl, (C₁-C₆)-alkoxy, (C₁-C₆)-alkylcarbonylamino, carboxyl, (C₁-C₆)-alkoxycarbonyl, mono- or di(C₁-C₆)-alkylamino, mono- or di(C₁-C₆)-alkylaminocarbonyl,

(C₁-C₆)-alkylsulfoxy, (C₁-C₆)-alkylsulfonyl, (C₆-C₁₀)-arylsulfoxy, (C₆-C₁₀)-arylsulfonyl, (C₁-C₆)-alkylaminosulfonyl, (C₁-C₆)-dialkylaminosulfonyl, hydroxymethyl, trifluoromethyl, trifluoromethoxy, halogen, hydroxyl, amino, nitro and (C₆-C₁₀)-aryl,

where (C₆-C₁₀)-aryl may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-lkyl, halo-(C₁-C₆)-lkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl,

R⁴ is hydrogen, (C₁-C₆)-acyl, (C₂-C₆)-alkenyl, (C₃-C₈)-cycloalkyl, or

R⁴ is (C₁-C₆)-alkyl which may be optionally substituted by 1 to 3 substituents which are selected from the group consisting of halogen, hydroxyl, (C₃-C₈)-cycloalkyl, (C₁-C₆)-acyl, (C₁-C₆)-alkoxy, carboxyl,

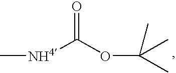

in which R^{4′} is hydrogen,

—(OCH₂CH₂)ₙOCH₂CH₃ in which n is 0 or 1, phenoxy, (C₆-C₁₀)-aryl and —NR¹³R¹⁴,

in which R¹³ and R¹⁴ are identical or different and are hydrogen, (C₁-C₆)-acyl, (C₁-C₆)-alkyl, carbamoyl, mono- or di(C₁-C₆)-alkylamino(C₁-C₆)-alkyl, mono- or di(C₁-C₆)-alkylaminocarbonyl, (C₆-C₁₀)-aryl or (C₁-C₆)-alkoxycarbonyl or

R¹³ and R¹⁴ form together with the nitrogen atom a 5- to 6-membered saturated heterocycle which may optionally comprise a further heteroatom from the series S or O or a radical of the formula —NR¹⁵, and may be substituted by oxo,

in which

R¹⁵ is hydrogen or (C₁-C₄)-alkyl, or

R⁴ is (C₁-C₆)-alkyl which is substituted by a 5- to 6-membered aromatic, optionally benzo-fused heterocycle having up to 3 heteroatoms from the series S, N and/or O, where a nitrogen-containing heterocycle may also be bonded via the nitrogen atom, or is substituted by

radicals of the formulae

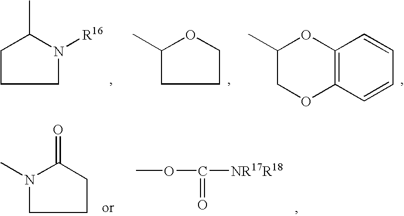

in which

R¹⁶ is hydrogen or (C₁-C₆)-alkyl,

R¹⁷ and R¹⁸ are identical or different and are hydrogen, (C₁-C₆)-alkyl or (C₆-C₁₀)-aryl, where aforementioned (C₁-C₆)-alkyl and (C₆-C₁₀)-aryl may optionally be substituted by 1 to 3 substituents selected from the group consisting of hydroxyl, (C₁-C₆)-alkoxy and halogen,

R⁵ is hydrogen, (C₁-C₆)-alkyl, halogen, amino, mono- or di(C₁-C₆)-alkylamino or is (C₁-C₆)-alkanoylamino,

R⁶ is phenyl which may optionally be substituted by one to three substituents selected from the group consisting of

halogen,

(C₆-C₁₀)-aryl which may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-lkyl, halo-(C₁-C₆)-alkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl, tri-(C₁-C₆)-alkylsilyloxy, a 3- to 8-membered, saturated or unsaturated, nonaromatic, mono- or bicyclic heterocycle which is optionally bonded via a nitrogen atom and has up to 3 heteroatoms from the series S, N and/or O, and/or cyano,

(C₁-C₆)-alkoxy,

(C₁-C₆)-alkoxycarbonyl,

(C₁-C₆)-alkylthio,

hydroxyl,

carboxyl,

partially fluorinated (C₁-C₆)-alkoxy having up to 6 fluorine atoms,

(C₁-C₆)-alkyl which is optionally substituted by a radical of the formula

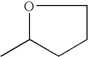

a 5- to 6-membered aromatic heterocycle which is optionally bonded via a nitrogen atom and has up to 3 heteroatoms from the series S, N and/or O and which may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-alkyl, halo-(C₁-C₆)-alkoxy, amino, (C₁-C₆)-alkylthio, hydroxyl, carboxyl, carbamoyl, aminocarbonyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl, a 3- to 8-membered saturated or unsaturated, nonaromatic, mono- or bicyclic heterocycle which is optionally bonded via a nitrogen atom and has up to 3 heteroatoms from the series S, N and/or O, and/or cyano,

a 3- to 8-membered saturated or unsaturated, nonaromatic, mono- or bicyclic heterocycle which is optionally bonded via a nitrogen atom and has up to 3 heteroatoms from the series S, N and/or O, and which may optionally be substituted by 1 to 3 substituents selected from oxo, halogen, hydroxyl, (C₁-C₆)-alkoxycarbonyl, (C₁-C₆)-alkoxycarbonylamino, (C₁-C₆)-alkyl, halo-(C₁-C₆)-alkyl and hydroxy-(C₁-C₆)-alkyl,

(C₂-C₆)-alkenyl

and groups of the formulae

OR¹⁹,

—NR²⁰R²¹ or —CO—NR²²R²³,

carbazole, dibenzofuran or dibenzothiophene,

xanthene or 9,10-dihydroacridine,

in which R¹⁹ is phenyl which in turn is optionally substituted by a group of the formula —NR²⁴R²⁵,

in which

R²⁴ and R²⁵ are identical or different and are hydrogen, (C₁-C₆)-alkyl or (C₁-C₆)-acyl,

or

R¹⁹ is (C₁-C₆)-alkyl which is optionally substituted once to three times by hydroxyl and/or halogen,

R²⁰ and R²¹ are identical or different and are hydrogen, carbamoyl, mono- or di-(C₁-C₆)-alkylaminocarbonyl, phenyl, (C₁-C₆)-acyl or (C₁-C₆)-alkyl,

where aforementioned (C₁-C₆)-alkyl is optionally substituted by (C₁-C₆)-alkoxy, (C₁-C₆)-acyl, by phenyl or by a 5- to 6-membered aromatic heterocycle having up to 3 heteroatoms from the series S, N and/or O,

where aforementioned phenyl and aforementioned aromatic heterocycle are optionally substituted once to three times, identically or differently, by halogen and/or hydroxyl, and

R²² and R²³ are identical or different and are hydrogen or (C₁-C₆)-alkyl,

and R⁷ may have the meaning of R⁵ and may be identical to or different from the latter,

and the salts thereof.

Physiologically acceptable salts of the compounds of the invention may be for example salts of the substances of the invention with mineral acids, carboxylic acids or sulfonic acids. Particularly preferred examples are salts with hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, citric acid, fumaric acid, maleic acid or benzoic acid.

Further salts which may be mentioned are salts with conventional bases, such as, for example, alkali metal salts (e.g. sodium or potassium salts), alkaline earth metal salts (e.g. calcium or magnesium salts) or ammonium salts derived from ammonia or organic amines such as, for example, diethylamine, triethylamine, ethyldiisopropylamine, procaine, dibenzylamine, N-methylmorpholine, dihydroabietylamine, 1-ephenamine or methylpiperidine.

The compounds of the invention may, depending on the substitution pattern, exist in stereoisomeric forms which either are related as image and mirror image (enantiomers), or are not related as image and mirror image (diastereomers). The invention relates either to the enantiomers or diastereomers or respective mixtures thereof. The racemic forms can, just like the diastereomers, be separated in a known manner into the stereoisomerically pure constituents.

The invention also includes within its scope compounds which are converted only in the body into the actual active ingredients of the formula (I) (so-called prodrugs).

(C₁-C₆)-Alkyl is expediently a straight-chain or branched alkyl radical having 1 to 6 carbon atoms. A straight-chain or branched alkyl radical having 1 to 4 carbon atoms (C₁-C₄) is preferred. Examples which may be mentioned are:

methyl, ethyl, n-propyl, isopropyl, n-butyl, sec-butyl, tert-butyl, n-pentyl and n-hexyl. A straight-chain or branched alkyl radical having 1 to 3 carbon atoms ((C₁-C₃)-alkyl) is particularly preferred.

Halo(C₁-C₆)-alkyl is expediently a (C₁-C₆)-alkyl group which may be defined as above and which has 1 to 3 halogen atoms, namely F, Cl, Br and/or I, preferably chlorine or fluorine as substituents, examples which may be mentioned being trifluoromethyl, fluoromethyl etc.

Hydroxy(C₁-C₆)-alkyl is expediently a (C₁-C₆)-alkyl group which may be defined as above and which has 1 to 3 hydroxyl groups as substituents, examples which may be mentioned being hydroxymethyl etc.

(C₂-C₆)-Alkenyl is for the purposes of the invention expediently a straight-chain or branched alkenyl radical having 2 to 6 carbon atoms. Examples which may be mentioned are: ethenyl, n-prop-2-en-1-yl and n-but-2-en-1-yl. A straight-chain or branched alkenyl radical having 2 to 4 carbon atoms is preferred.

(C₁-C₆)-Alkoxy is expediently a straight-chain or branched alkoxy radical having 1 to 6 carbon atoms. A straight-chain or branched alkoxy radical having 1 to 4 carbon atoms (C₁-C₄) is preferred. Examples which may be mentioned are: methoxy, ethoxy, n-propoxy, isopropoxy, tert-butoxy, n-pentoxy and n-hexoxy. A straight-chain or branched alkoxy radical having 1 to 3 carbon atoms (C₁-C₃) is particularly preferred.

Halo-(C₁-C₆)-alkoxy is expediently mono- or polyhalo-substituted (C₁-C₆)-alkoxy. Reference may be made to the above definition concerning the (C₁-C₆)-alkoxy portion, and the definition of halogen. For example, halo-(C₁-C₆)-alkoxy includes (C₁-C₆)-alkoxy which is partially chlorinated and/or fluorinated one or more times, or perfluorinated, such as trifluoromethoxy, fluoromethoxy, chloromethoxy, pentafluoroethoxy, trifluoromethylmethoxy etc.

Partially fluorinated (C₁-C₆)-alkoxy having up to 6 fluorine atoms is expediently a straight-chain or branched alkoxy radical which has 1 to 6 carbon atoms and which may be substituted by 1 to 6, preferably 1 to 4, more preferably 1 to 3 fluorine atoms. A straight-chain or branched alkoxy radical having 1 to 4 carbon atoms and 1 to 4 fluorine atoms is preferred. Examples which may be mentioned are: methoxy, ethoxy, n-propoxy, isopropoxy, tert-butoxy, n-pentoxy and n-hexoxy, each of which has 1 to 4 fluorine atoms. (1,3-Difluoroprop-2-yl)oxy and 1,1,2,2-tetrafluoroethoxy are particularly preferred.

(C₁-C₆)-Alkylthio is expediently a straight-chain or branched alkylthio radical having 1 to 6 carbon atoms. A straight-chain or branched alkylthio radical having 1 to 4 carbon atoms (C₁-C₄) is preferred. Examples which may be mentioned are: methylthio, ethylthio, n-propylthio, isopropylthio, tert-butylthio, n-pentylthio and n-hexylthio. A straight-chain or branched alkylthio radical having 1 to 3 carbon atoms (C₁-C₃)-alkylthio is particularly preferred.

(C₁-C₆)-Alkoxycarbonyl is expediently a straight-chain or branched alkoxycarbonyl radical having 1 to 6 carbon atoms. A straight-chain or branched alkoxycarbonyl radical having 1 to 4 carbon atoms (C₁-C₄) is preferred. Examples which may be mentioned are: methoxycarbonyl, ethoxycarbonyl, n-propoxycarbonyl, isopropoxycarbonyl and tert-butoxycarbonyl. A straight-chain or branched alkoxycarbonyl radical having 1 to 4 carbon atoms (C₁-C₄) is particularly preferred.

Mono- or di-(C₁-C₆)-alkylaminocarbonyl expediently for the purposes of the invention is expediently a carbamoyl group (H₂N—CO—), in which one or both hydrogen atoms are replaced by a (C₁-C₆)-alkyl group. Concerning the definition of the (C₁-C₆)-alkyl group, reference may be made to the above explanation of (C₁-C₆)-alkyl. Methylaminocarbonyl, dimethylaminocarbonyl etc. may be mentioned as examples.

Mono- or di-(C₁-C₆)-acylamino for the purposes of the invention is expediently an amino group (H₂N—), in which one or both hydrogen atoms are replaced by a (C₁-C₆)-acyl group. Concerning the definition of the (C₁-C₆)-acyl group, reference may be made to the explanation of (C₁-C₆)-acyl above. (C₁-C₆)-Alkanoyl as mentioned in the definition of (C₁-C₆)-acyl may be mentioned by way of example.

(C₁-C₆)-Alkylsulfoxy is expediently a (C₁-C₆)-alkyl-S(═O) group and, concerning the (C₁-C₆)-alkyl group, reference may be made to the definition thereof above.

(C₁-C₆)-Alkylsulfonyl is expediently a (C₁-C₆)-alkyl-SO₂ group and, concerning the (C₁-C₆)-alkyl group, reference may be made to the definition thereof above.

(C₆-C₁₀)-Aryl is generally an aromatic radical having 6 to 10 carbon atoms. Preferred aryl radicals are phenyl and naphthyl.

(C₁-C₆)-Acyl is for the purposes of the invention expediently a straight-chain or branched acyl radical having 1 to 6 carbon atoms. Examples which may be mentioned are: formyl, acetyl, ethanoyl, propanoyl, isopropanoyl, butanoyl, isobutanoyl and pentanoyl. A straight-chain or branched acyl radical having 1 to 4 carbon atoms is preferred. Acetyl and ethanoyl are particularly preferred.

(C₃-C₈)-Cycloalkyl is for the purposes of the invention cyclopropyl, cyclopentyl, cyclobutyl, cyclohexyl, cycloheptyl or cyclooctyl. Those which may be mentioned as preferred are: cyclopropyl, cyclopentyl or cyclohexyl. The meaning of (C₃-C₆)-cycloalkyl correspondingly is expediently cyclopropyl, cyclopentyl, cyclobutyl, cyclohexyl.

Halogen is for the purposes of the invention generally fluorine, chlorine, bromine and iodine. Fluorine, chlorine and bromine are preferred. Fluorine and chlorine are particularly preferred.

(C₁-C₆)-Alkanoyl is for the purposes of the invention formyl and (C₁-C₅)-alkylcarbonyl groups, (C₁-C₅)-alkyl may be a straight-chain or branched-chain alkyl group having 1 to 5 carbon atoms, for example acetyl, propionyl, butyryl, pentanoyl.

A 5- to 6-membered aromatic heterocycle having up to 3 heteroatoms from the series S, O and/or N is, for example, pyridyl, pyrimidyl, thienyl, furyl, pyrrolyl, thiazolyl, N-triazolyl, oxazolyl or imidazolyl. Pyridyl, furyl, thiazolyl and N-triazolyl are preferred.

A 5- to 6-membered aromatic benzo-fused heterocycle having up to 3 heteroatoms from the series S, O and/or N is, for example, benzimidazolyl.

A 5- to 6-membered saturated heterocycle which is bonded via a nitrogen atom, which may be formed from two substituent groups together with the nitrogen atom to which they are bonded, and which may optionally contain a further heteroatom from the series S or O or a radical of the formula —NR¹⁵ in which R¹⁵ is as defined above, is for the purposes of the invention generally morpholinyl, piperidinyl, piperazinyl, methylpiperazinyl, thiomorpholinyl or pyrrolidinyl. Morpholinyl, piperidinyl, pyrrolidinyl and thiomorpholinyl are particularly preferred.

A 3- to 8-membered saturated or unsaturated, nonaromatic heterocycle which is optionally bonded via a nitrogen atom and has up to 3 heteroatoms from the series S, N and/or O includes for example the abovementioned 5- to 6-membered saturated heterocycles bonded via a nitrogen atom, and 3-, 7- and 8-membered heterocycles such as, for example, aziridines (e.g. 1-azacyclopropan-1-yl), azetidines (e.g. 1-azacyclobutan-1-yl) and azepines (e.g. 1-azepan-1-yl). The unsaturated representatives may comprise 1 to 2 double bonds in the ring.

The side group of a naturally occurring α-amino acid in the meaning of R¹⁰ includes for example: hydrogen (glycine), methyl (alanine), propan-2-yl (valine), 2-methylpropan-1-yl (leucine), 1-methylpropan-1-yl (isoleucine), a propane-1,3-diyl group which is connected to the nitrogen atom of the amino group (proline), a 2-hydroxypropane-1,3-diyl group which is connected to the nitrogen atom of the amino group (hydroxyproline), a group of the formula

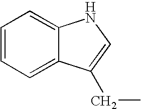

(tryptophan), a benzyl group (phenylalanine), a methylthioethyl group (methionine), hydroxymethyl (serine), p-hydroxybenzyl (tyrosine), 1-hydroxyethan-1-yl (threonine), mercaptomethyl (cysteine), carbamoylmethyl (asparagine), carbamoylethyl (glutamine), carboxymethyl (aspartic acid), carboxyethyl (glutamic acid), 4-aminobutan-1-yl (lysine), 3-guanidinopropan-1-yl (arginine), imidazol-4-ylmethyl (histidine), 3-ureidopropan-1-yl (citrulline), mercaptoethyl (homocysteine), hydroxyethyl (homoserine), 4-amino-3-hydroxybutan-1-yl (hydroxylysine), 3-aminopropan-1-yl (ornithine), etc.

The present invention further relates to compounds of the general formula (I)

in which

R¹ is hydrogen, halogen, (C₁-C₆)-alkyl, (C₁-C₆)-alkoxy, amino-(C₁-C₆)-alkyl or halo-(C₁-C₆)-alkyl,

R² is hydrogen, (C₃-C₈)-cycloalkyl or biphenylaminocarbonyl, or

is (C₁-C₆)-alkyl which is optionally substituted by 1 to 3 substituents which are selected from the group consisting of (C₃-C₆)-cycloalkyl, (C₁-C₆)-alkoxy, halogen, hydroxyl, amino, tri-(C₁-C₆)-alkylsilyoxy, radicals of the formula

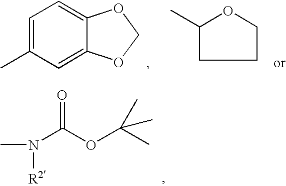

in which R^{2′} is hydrogen or (C₁-C₄)-alkyl,

a 5- to 6-membered aromatic heterocycle having up to 3 heteroatoms from the series S, N and/or O, where a nitrogen-containing heterocycle may also be bonded via the nitrogen atom,

a 3- to 8-membered saturated or unsaturated, nonaromatic, heterocycle which is optionally bonded via a nitrogen atom and has up to 3 heteroatoms from the series S, N and/or O, and

(C₆-C₁₀)-aryl which may in turn be substituted by hydroxyl or (C₁-C₆)-alkoxy, or

is a group of the formula

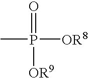

in which R⁸ and R⁹ are identical to or different from one another and are hydrogen and (C₁-C₄)-alkyl, or

are a group of the formula

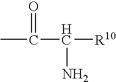

in which R¹⁰ is the side group of a naturally occurring α-amino acid, or

is a group of the formula

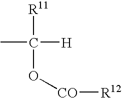

in which R¹¹ is (C₁-C₄)-alkyl, and R¹² is hydrogen, (C₁-C₄)-alkyl or is a group of the formula

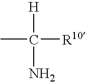

in which R^{10′} is the side group of a naturally occurring α-amino acid,

or in which

R² is (C₃-C₈)-cycloalkyl

which is optionally substituted by 1 to 3 substituents selected from the group consisting of (C₁-C₆)-alkyl, (C₃-C₆)-cycloalkyl, (C₁-C₆)-alkoxy, trifluoromethyl, trifluoromethoxy, halogen, hydroxyl, amino and nitro,

R³ is (C₃-C₈)-cycloalkyl

which is substituted by 1 to 3 substituents selected from the group consisting of (C₁-C₆)-alkyl, (C₃-C₆)-cycloalkyl, (C₁-C₆)-alkoxy, (C₁-C₆)-alkylcarbonylamino, carboxyl, (C₁-C₆)-alkoxycarbonyl, mono- or di-(C₁-C₆)-alkylamino, mono- or di-(C₁-C₆)-alkylaminocarbonyl, (C₁-C₆)-alkylsulfoxy, (C₁-C₆)-alkylsulfonyl, (C₆-C₁₀)-arylsulfoxy, (C₆-C₁₀)-arylsulfonyl, (C₁-C₆)-alkylaminosulfonyl, (C₁-C₆)-dialkylaminosulfonyl, hydroxymethyl, trifluoromethyl, trifluoromethoxy, halogen, hydroxyl, amino, nitro and (C₆-C₁₀)-aryl,

where (C₆-C₁₀)-aryl may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-lkyl, halo-(C₁-C₆)-lkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl,

R⁴ is hydrogen, (C₁-C₆)-acyl, (C₂-C₆)-alkenyl, (C₃-C₈)-cycloalkyl, or

R⁴ is (C₁-C₆)-alkyl which may optionally be substituted by 1 to 3 substituents which are selected from the group consisting of halogen, hydroxyl, (C₃-C₈)-cycloalkyl, (C₁-C₆)-acyl, (C₁-C₆)-alkoxy, carboxyl,

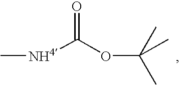

in which R^{4′} is hydrogen,

—(OCH₂CH₂)ₙOCH₂CH₃ in which n is 0 or 1, phenoxy, (C₆-C₁₀)-aryl and —NR¹³R¹⁴,

in which R¹³ and R¹⁴ are identical or different and are hydrogen, (C₁-C₆)-acyl, (C₁-C₆)-alkyl, carbamoyl, mono- or di(C₁-C₆)-alkylamino(C₁-C₆)-alkyl, mono- or di(C₁-C₆)-alkylaminocarbonyl, (C₆-C₁₀)-aryl or (C₁-C₆)-alkoxycarbonyl, or

R¹³ and R¹⁴ form together with the nitrogen atom a 5- to 6-membered saturated heterocycle which may optionally comprise a further heteroatom from the series S or O or a radical of the formula —NR¹⁵, and may be substituted by oxo,

in which

R¹⁵ is hydrogen or (C₁-C₄)-alkyl, or

R⁴ is (C₁-C₆)-alkyl which is substituted by a 5- to 6-membered aromatic, optionally benzo-fused heterocycle having up to 3 heteroatoms from the series S, N and/or O, where a nitrogen-containing heterocycle may also be bonded via the nitrogen atom, or is substituted by

radicals of the formulae

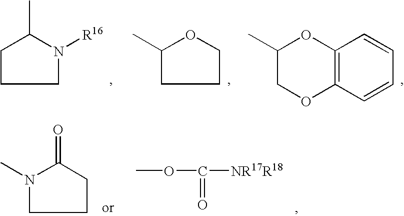

in which

R¹⁶ is hydrogen or (C₁-C₆)-alkyl,

R¹⁷ and R¹⁸ are identical or different and are hydrogen, (C₁-C₆)-alkyl or (C₆-C₁₀)-aryl, where aforementioned (C₁-C₆)-alkyl and (C₆-C₁₀)-aryl may optionally be substituted by 1 to 3 substituents selected from the group consisting of hydroxyl, (C₁-C₆)-alkoxy and halogen,

R⁵ is hydrogen, (C₁-C₆)-alkyl, halogen, amino, mono- or di(C₁-C₆)-alkylamino or is (C₁-C₆)-alkanoylamino,

R⁶ is phenyl which may optionally be substituted by one to three substituents selected from the group consisting of

halogen,

(C₆-C₁₀)-aryl which may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-lkyl, halo-(C₁-C₆)-alkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonyl amino, (C₁-C₆)-alkylsulfoxy, (C₁-C₆)-alkylsulfonyl, tri-(C₁-C₆)-alkylsilyloxy, a 3- to 8-membered, saturated or unsaturated, nonaromatic, mono- or bicyclic heterocycle which is optionally bonded via a nitrogen atom and has up to 3 heteroatoms from the series S, N and/or O, and/or cyano,

(C₁-C₆)-alkoxy,

(C₁-C₆)-alkoxycarbonyl,

(C₁-C₆)-alkylthio,

hydroxyl,

carboxyl,

partially fluorinated (C₁-C₆)-alkoxy having up to 6 fluorine atoms,

(C₁-C₆)-alkyl which is optionally substituted by a radical of the formula

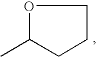

a 5- to 6-membered aromatic heterocycle which is optionally bonded via a nitrogen atom and carries up to 3 heteroatoms from the series S, N and/or O and which may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-alkyl, halo-(C₁-C₆)-alkoxy, amino, (C₁-C₆)-alkylthio, hydroxyl, carboxyl, carbamoyl, aminocarbonyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl, a 3- to 8-membered saturated or unsaturated, nonaromatic, mono- or bicyclic heterocycle which is optionally bonded via a nitrogen atom and has up to 3 heteroatoms from the series S, N and/or O, and/or cyano,

a 3- to 8-membered saturated or unsaturated, nonaromatic, mono- or bicyclic heterocycle which is optionally bonded via a nitrogen atom and has up to 3 heteroatoms from the series S, N and/or O, and which may optionally be substituted by 1 to 3 substituents selected from oxo, halogen, hydroxyl, (C₁-C₆)-alkoxycarbonyl, (C₁-C₆)-alkoxycarbonylamino, (C₁-C₆)-alkyl, halo-(C₁-C₆)-alkyl and hydroxy-(C₁-C₆)-alkyl,

(C₂-C₆)-alkenyl,

and groups of the formulae

OR¹⁹,

—NR²⁰R²¹ or —CO—NR²²R²³,

carbazole, dibenzofuran or dibenzothiophene,

xanthene or 9,10-dihydroacridine,

in which R¹⁹ is phenyl which in turn is optionally substituted by a group of the formula —NR²⁴R²⁵,

in which

R²⁴ and R²⁵ are identical or different and are hydrogen, (C₁-C₆)-alkyl or (C₁-C₆)-acyl,

or

R¹⁹ is (C₁-C₆)-alkyl which is optionally substituted once to three times by hydroxyl and/or halogen,

R²⁰ and R²¹ are identical or different and are hydrogen, carbamoyl, mono- or di-(C₁-C₆)-alkylaminocarbonyl, phenyl, (C₁-C₆)-acyl or (C₁-C₆)-alkyl,

where aforementioned (C₁-C₆)-alkyl is optionally substituted by (C₁-C₆)-alkoxy, (C₁-C₆)-acyl, by phenyl or by a 5- to 6-membered aromatic heterocycle having up to 3 heteroatoms from the series S, N and/or O,

where aforementioned phenyl and aforementioned aromatic heterocycle are optionally substituted once to three times, identically or differently, by halogen and/or hydroxyl, and

R²² and R²³ are identical or different and are hydrogen or (C₁-C₆)-alkyl,

and R⁷ may have the meaning of R⁵ and may be identical to or different from the latter,

and the salts thereof.

In a further preferred embodiment, the invention relates to compounds of the general formula (I) in which

R¹ is hydrogen or (C₁-C₆)-alkyl,

R² is hydrogen, or

is (C₁-C₆)-alkyl which is optionally substituted by 1 to 3 substituents selected from the group [lacuna] of (C₃-C₆)-cycloalkyl, (C₁-C₆)-alkoxy, halogen, hydroxyl, amino,

R² is (C₃-C₈)-cycloalkyl

which is optionally substituted by 1 to 3 substituents selected from the group consisting of (C₁-C₆)-alkyl, halogen, hydroxyl and amino,

R³ is (C₃-C₈)-cycloalkyl

which is substituted by 1 to 3 substituents selected from the group consisting of (C₁-C₆)-alkyl, (C₃-C₆)-cycloalkyl, (C₁-C₆)-alkoxy, halogen, hydroxyl, amino and (C₆-C₁₀)-aryl,

where (C₆-C₁₀)-aryl may optionally be substituted by 1 to 3 substituents selected from (C₁-C₆)-alkoxy, (C₁-C₆)-alkyl, halogen, amino, hydroxyl, carboxyl,

R⁴ is hydrogen, or

R⁴ is (C₁-C₆)-alkyl which may optionally be substituted by 1 to 3 substituents selected from the group consisting of halogen and hydroxyl,

R⁵ is hydrogen or (C₁-C₆)-alkyl,

R⁶ is phenyl which may optionally be substituted by one to three substituents selected from the group consisting of

halogen,

(C₆-C₁₀)-aryl which may optionally be substituted by 1 to 2 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-alkyl, halo-(C₁-C₆)-alkoxy, amino, hydroxyl, carboxyl, carbamoyl, mono- or di-(C₁-C₆)-alkylaminocarbonyl, mono- or di-(C₁-C₆)-alkanoylamino, a 3- to 8-membered saturated or unsaturated, nonaromatic, mono- or bicyclic heterocycle which is optionally bonded via a nitrogen atom and has up to 3 heteroatoms from the series S, N and/or O, and/or cyano,

(C₁-C₆)-alkoxy,

(C₁-C₆)-alkoxycarbonyl,

(C₁-C₆)-alkylthio

hydroxyl,

carboxyl,

partially fluorinated (C₁-C₆)-alkoxy having up to 6 fluorine atoms,

a 5- to 6-membered aromatic heterocycle which is optionally bonded via a nitrogen atom and has up to 3 heteroatoms from the series S, N and/or O and which may optionally be substituted by 1 to 2 substituents selected from (C₁-C₆) alkanoyl, (C₁-C₆)-alkoxy, (C₁-C₆)-alkyl, halogen, (C₁-C₆)-alkoxycarbonyl, nitro, halo-(C₁-C₆)-alkyl, halo-(C₁-C₆)-alkoxy, amino, hydroxyl, carboxyl, aminocarbonyl, mono- or di-(C₁-C₆)-alkylaminocarbonyl, mono- or di-(C₁-C₆)-alkanloylamino, a 3- to 8-membered saturated or unsaturated, nonaromatic, mono- or bicyclic heterocycle which is optionally bonded via a nitrogen atom and has up to 3 heteroatoms from the series S, N and/or O and/or cyano,

a 3- to 8-membered saturated or unsaturated, nonaromatic, mono- or bicyclic heterocycle which is optionally bonded via a nitrogen atom and has up to 3 heteroatoms from the series S, N and/or O, and which may optionally be substituted by 1 to 2 substituents selected from oxo, halogen, hydroxyl, (C₁-C₆)-alkoxycarbonyl, (C₁-C₆)-alkyl, halo-(C₁-C₆)-alkyl and hydroxy-(C₁-C₆)-alkyl,

(C₂-C₆)-alkenyl,

and R⁷ may have the meaning of R⁵ and be identical to or different from the latter,

and the salts thereof.

In a further preferred embodiment, the invention relates to compounds of the general formula (I) in which

R¹ is hydrogen or (C₁-C₆)-alkyl,

R² is hydrogen, or

is (C₁-C₆)-alkyl, which is optionally substituted by 1 to 3 substituents selected from the group consisting of (C₃-C₆)-cycloalkyl, (C₁-C₆)-alkoxy, halogen, hydroxyl and amino,

R² is (C₃-C₈)-cycloalkyl,

which is optionally substituted by 1 to 3 substituents selected from the group consisting of (C₁-C₆)-alkyl, halogen, hydroxyl and amino,

R³ is (C₃-C₈)-cycloalkyl

which is substituted by 1 to 3 substituents selected from the group consisting of (C₁-C₆)-alkyl, (C₃-C₆)-cycloalkyl, (C₁-C₆)-alkoxy, halogen, hydroxyl, amino and (C₆-C₁₀)-aryl,

where (C₆-C₁₀)-aryl may optionally be substituted by 1 to 3 substituents selected from (C₁-C₆)-alkoxy, (C₁-C₆)-alkyl, halogen, amino, hydroxyl, carboxyl,

R⁴ is hydrogen, or

R⁴ is (C₁-C₆)-alkyl, which may optionally be substituted by 1 to 3 substituents selected from the group consisting of halogen and hydroxyl,

R⁵ is hydrogen or (C₁-C₆)-alkyl,

R⁶ is phenyl which may optionally be substituted by one to three substituents selected from the group consisting of

halogen,

(C₆-C₁₀)-aryl, which may optionally be substituted by 1 to 2 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-alkyl, halo-(C₁-C₆)-alkoxy, amino, hydroxyl, carboxyl, carbamoyl, mono- or di-(C₁-C₆)-alkylaminocarbonyl, mono- or di-(C₁-C₆)-alkanoylamino, a 3- to 8-membered saturated or unsaturated, nonaromatic, mono- or bicyclic heterocycle which is optionally bonded by a nitrogen atom and has up to 3 heteroatoms from the series of S, N and/or O, and/or cyano,

(C₁-C₆)-alkoxy,

(C₁-C₆)-alkoxycarbonyl,

(C₁-C₆)-alkylthio,

hydroxyl,

carboxyl,

partially fluorinated (C₁-C₆)-alkoxy having up to 6 fluorine atoms,

a 5- to 6-membered aromatic heterocycle which is optionally bonded via a nitrogen atom and has up to 3 heteroatoms from the series S, N and/or O and which may optionally be substituted by 1 to 2 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-alkyl, halo-(C₁-C₆)-alkoxy, amino, hydroxyl, carboxyl, aminocarbonyl, mono- or di-(C₁-C₆)-alkylaminocarbonyl, mono- or di-(C₁-C₆)-alkanoylamino, a 3- to 8-membered saturated or unsaturated, nonaromatic, mono- or bicyclic heterocycle which is optionally bonded via a nitrogen atom and has up to 3 heteroatoms from the series S, N and/or O, and/or cyano,

a 3- to 8-membered saturated or unsaturated, nonaromatic, mono- or bicyclic heterocycle which is optionally bonded via a nitrogen atom and has up to 3 heteroatoms from the series S, N and/or O and which may optionally be substituted by 1 to 2 substituents selected from oxo, halogen, hydroxyl, (C₁-C₆)-alkoxycarbonyl, (C₁-C₆)-alkyl, halo-(C₁-C₆)-alkyl and hydroxy-(C₁-C₆)-alkyl,

(C₂-C₆)-alkenyl,

and R⁷ may have the meaning of R⁵ and be identical to or different from the latter,

and the salts thereof.

In a preferred embodiment, the invention relates to compounds of the general formula (I) in which R¹ is hydrogen or (C₁-C₆)-alkyl, in particular methyl.

In a further preferred embodiment, the invention relates to compounds of the general formula (I) in which R² is hydrogen or (C₁-C₆)-alkyl, in particular methyl.

In a further preferred embodiment, the invention relates to compounds of the general formula (I) in which R³ is cyclopropyl which is substituted by 1 to 2, in particular one substituent selected independently of one another from the group consisting of phenyl, (C₁-C₃)-alkyl, in particular methyl and halogen, in particular fluorine.

In a further preferred embodiment, the invention relates to compounds of the general formula (I) in which R⁴ is hydrogen or (C₁-C₆)-alkyl, in particular methyl.

In a further preferred embodiment, the invention relates to compounds of the general formula (I) in which R⁵ is hydrogen.

In a further preferred embodiment, the invention relates to compounds of the general formula (I) in which R⁶ is phenyl which may optionally be substituted by one to three substituents selected from the group consisting of

halogen,

(C₆-C₁₀)-aryl which may optionally be substituted by 1 to 3 substituents selected from (C₁-C₆)alkanoyl, (C₁-C₆)-alkoxy, (C₁-C₆)-alkyl, halogen, (C₁-C₆)alkoxycarbonyl, nitro, halo-(C₁-C₆)alkyl, halo(C₁-C₆)alkoxy, amino, (C₁-C₆)alkylthio, hydroxyl, carboxyl, carbamoyl, mono- or di(C₁-C₆)alkylaminocarbonyl, mono- or di(C₁-C₆)alkanoylamino, (C₁-C₆)alkoxycarbonylamino, (C₁-C₆)alkylsulfoxy, (C₁-C₆)alkylsulfonyl, tri(C₁-C₆)alkylsilyloxy, a 3- to 8-membered saturated or unsaturated, nonaromatic, mono- or bicyclic heterocycle which is optionally bonded via a nitrogen atom and has up to 3 heteroatoms from the series S, N and/or O, and/or cyano,

(C₁-C₆)-alkoxy,

(C₁-C₆)-alkoxycarbonyl,

(C₁-C₆)-alkylthio,

hydroxyl,

carboxyl,

partially fluorinated (C₁-C₆)-alkoxy having up to 6 fluorine atoms,

(C₁-C₆)-alkyl which is optionally substituted by a radical of the formula

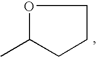

a 5- to 6-membered aromatic heterocycle which is optionally bonded via a nitrogen atom and has up to 3 heteroatoms from the series S, N and/or O and which may optionally be substituted by 1 to 3 substituents selected from (C₁-C₆)alkanoyl, (C₁-C₆)-alkoxy, (C₁-C₆)-alkyl, halogen, (C₁-C₆)alkoxycarbonyl, nitro, halo(C₁-C₆)alkyl, halo(C₁-C₆)alkoxy, amino, (C₁-C₆)alkylthio, hydroxyl, carboxyl, carbamoyl, mono- or di(C₁-C₆)alkylaminocarbonyl, mono- or di(C₁-C₆)alkanoylamino, (C₁-C₆)alkoxycarbonylamino, (C₁-C₆)alkylsulfoxy, (C₁-C₆)alkylsulfonyl, a 3- to 8-membered saturated or unsaturated, nonaromatic, mono- or bicyclic heterocycle which is optionally bonded via a nitrogen atom and has up to 3 heteroatoms from the series S, N and/or O, and/or cyano,

a 3- to 8-membered saturated or unsaturated, nonaromatic, mono- or bicyclic heterocycle which is optionally bonded via a nitrogen atom and has up to 3 heteroatoms from the series S, N and/or O and which may optionally be substituted by 1 to 3 substituents selected from oxo, halogen, hydroxyl, (C₁-C₆)-alkoxycarbonyl, (C₁-C₆)-alkoxycarbonyl-amino, (C₁-C₆)-alkyl, halo(C₁-C₆)alkyl and hydroxy(C₁-C₆)-alkyl,

and groups of the formulae

—OR¹⁹,

—NR²⁰R²¹ or —CO—NR²²R²³,

in which

R¹⁹ is phenyl which in turn is optionally substituted by a group of the formula —NR²⁴R²⁵,

in which

R²⁴ and R²⁵ are identical or different and are hydrogen, (C₁-C₆)-alkyl or (C₁-C₆)-acyl,

or

R¹⁹ is (C₁-C₆)-alkyl which is optionally substituted once to three times by hydroxyl and/or halogen,

R²⁰ and R²¹ are identical or different and are hydrogen, carbamoyl, mono- or di(C₁-C₆)-alkylaminocarbonyl, phenyl, (C₁-C₆)-acyl or (C₁-C₆)-alkyl,

where aforementioned (C₁-C₆)-alkyl is optionally substituted by (C₁-C₆)-alkoxy, (C₁-C₆)-acyl, by phenyl or by a 5- to 6-membered aromatic heterocycle having up to 3 heteroatoms from the series S, N and/or O,

where aforementioned phenyl and aforementioned aromatic heterocycle are optionally substituted once to three times, identically or differently, by halogen and/or hydroxyl, and

R²² and R²³ are identical or different and are hydrogen or (C₁-C₆)-alkyl, and [lacuna]

In a further preferred embodiment, the invention relates to compounds of the general formula (I) in which R⁷ is hydrogen.

The invention further relates to processes for preparing the compounds of the general formula (I), characterized in that

[A] compounds of the general formula (II)

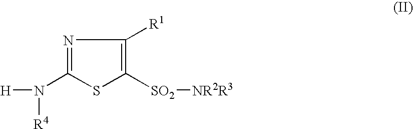

in which

R¹, R², R³ and R⁴ have the abovementioned meaning,

are reacted with compounds of the general formula (III)

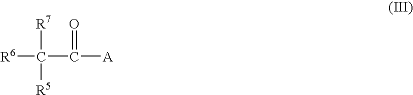

in which

A is a leaving group such as, for example, halogen, preferably chlorine, or hydroxyl, and R⁵, R⁶ and R⁷ have the abovementioned meaning, in inert solvents, where appropriate in the presence of a base and/or of an auxiliary to give compounds of the formula (I), or

[B] compounds of the general formula (IV)

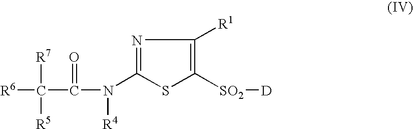

in which

R¹, R⁴, R⁵, R⁶ and R⁷ have the abovementioned meaning, and D is a halogen atom, preferably chlorine, are reacted with amines of the general formula (V):

HNR²R³ (V)

in which

R² and R³ have the abovementioned meaning, in inert solvents to give compounds of the formula (I), or

[C] compounds of the general formula (X)

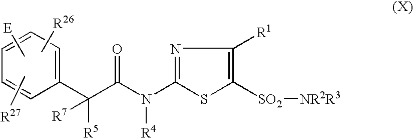

in which

R¹, R², R³, R⁴, R⁵, R⁷, R²⁶, and R²⁷ have the abovementioned meaning, and E is trifluoromethanesulfonate or halogen, preferably bromine or iodine, are reacted with boronic acids or stannanes of the general formula (XI):

R²⁸M (XI)

in which

R²⁸ has the abovementioned meaning, and M can be for example a tri(C₁-C₆)-alkylstannyl group, such as a trimethylstannyl group or a boronic acid group, in inert solvents in the presence of palladium catalysts, e.g. tetrakis(triphenylphosphane)palladium (0), where appropriate in the presence of base, e.g. potassium phosphate at temperatures of 50-140° C. to give compounds of the formula (XIV)

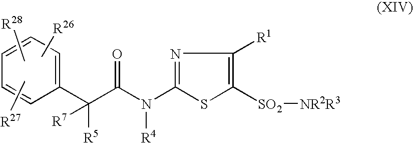

or

[D] compounds of the general formula (XII)

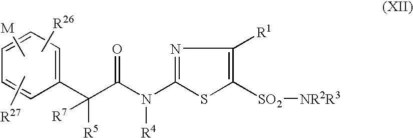

in which

R¹, R², R³, R⁴, R⁵, R⁷, R²⁶ and R²⁷ have the abovementioned meaning, and M has the abovementioned meaning, are reacted with trifluoromethanesulfonates or halides of the general formula (XIII):

R²⁸E (XIII)

in which

R²⁸ has the abovementioned meaning, and E has the abovementioned meaning, in inert solvents in the presence of palladium catalysts, e.g. tetrakis(triphenylphosphane)palladium(0), where appropriate in the presence of base, e.g. potassium phosphate at temperatures of 50-140° C. to give compounds of the formula (XIV).

Process [A] of the invention can be illustrated by way of example by the following formula diagram:

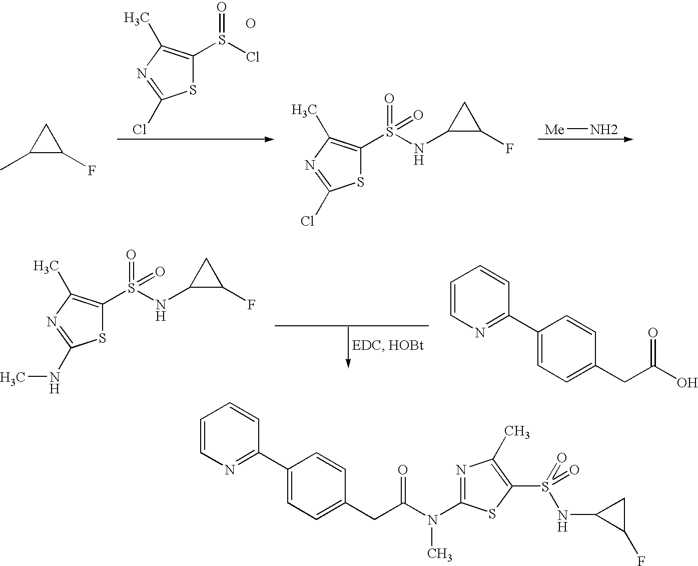

The meanings here are:

HOBt: 1-Hydroxy-1H-benzotriazole

EDC: N′-(3-Dimethylaminopropyl)-N-ethylcarbodiimide×HCl

DMF: N,N-Dimethylformamide

Process [C] of the invention can be illustrated by way of example by the following formula diagrams:

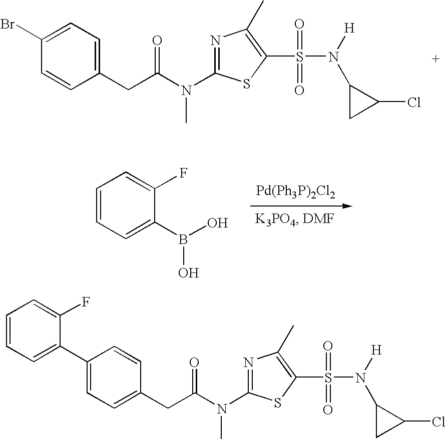

The meaning here is:

DMF: N,N-Dimethylformamide

Process [D] of the invention can be illustrated by way of example by the following formula diagrams:

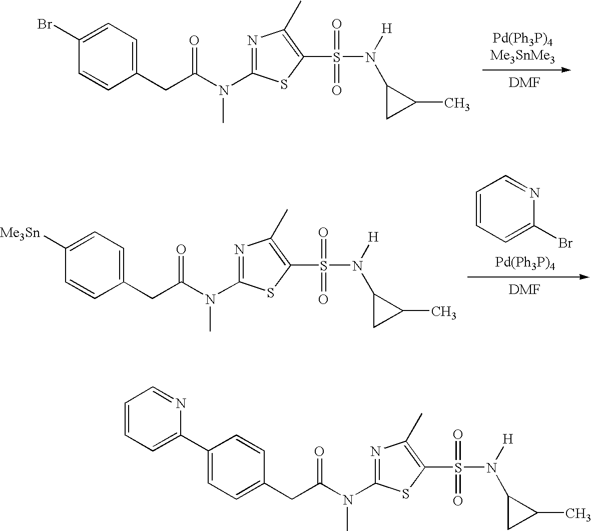

The meaning here is:

DMF: N,N-Dimethylformamide

Solvents suitable for processes [A], [B], [C] and [D] are conventional organic solvents which are not changed under the reaction conditions. These preferably include ethers such as diethyl ether, dioxane, tetrahydrofuran, glycol dimethyl ether, or hydrocarbons such as benzene, toluene, xylene, hexane, cyclohexane or petroleum fractions, or halohydrocarbons such as dichloromethane, trichloromethane, tetrachloromethane, dichloroethylene, trichloroethylene or chlorobenzene, or ethyl acetate, dimethyl sulfoxide, dimethyl formamide (DMF) or acetonitrile. It is likewise possible to use mixtures of said solvents. DMF is preferred.

Bases which can generally be employed for process [A] of the invention are inorganic or organic bases. These preferably include organic amines (trialkyl(C₁-C₆)amines) such as triethylamine, or heterocycles such as 1,4-diazabicyclo[2.2.2]octane (DABCO), 1,8-diazabicyclo[5.4.0]undec-7-ene (DBU), pyridine, diaminopyridine, N-methylmorpholine or N-methylpiperidine or morpholine. Triethylamine is preferred.

Suitable auxiliaries are dehydrating or coupling reagents known per se, such as, for example, carbodiimides such as diisopropylcarbodiimide, dicyclohexylcarbodiimide (DCC) or N-(3-dimethylaminopropyl)-N′-ethylcarbodiimide (EDC), or carbonyl compounds such as carbonyldiimidazole (CDI) or isobutyl chloroformate, or 1,2-oxazolium compounds such as 2-ethyl-5-phenyl-1,2-oxazolium-3-sulfonate, or phosphorus compounds such as propanephosphonic anhydride, diphenylphosphoric azide, benzotriazolyl-N-oxytris(dimethylamino)phosphonium hexafluorophosphate (BOP), or uronium compounds such as O-benzotriazol-1-yl-N,N′,N′-tetramethyluronium hexafluorophosphate (HBTU), or methanesulfonyl chloride, where appropriate in the presence of aids such as N-hydroxysuccinimide or N-hydroxybenzotriazole.

The base is generally employed in an amount of from 0.05 mol to 10 mol, preferably from 1 mol to 2 mol, based on 1 mol of the compound of the formula (III).

The processes of the invention are generally carried out in a temperature range from −50° C. to +100° C., preferably from −30° C. to +60° C.

The processes of the invention are generally carried out under atmospheric pressure. It is, however, also possible to carry out the process under elevated pressure or under reduced pressure (for example in a range from 0.5 to 5 bar).

The compounds of the general formula (II) can be prepared for example by converting compounds of the general formula (VI)

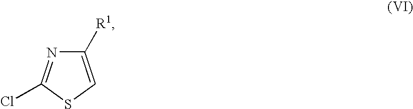

in which

R¹ has the abovementioned meaning,

by reaction with the chlorosulfonic acid/SOCl₂ system into the compounds of the general formula (VII)

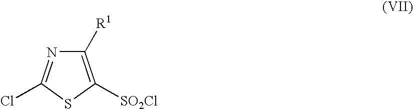

in which

R¹ has the abovementioned meaning,

subsequently preparing with amines of the general formula (V)

HNR²R³ (V)

in which

R² and R³ have the abovementioned meaning,

in inert solvents, the compounds of the general formula (VIII)

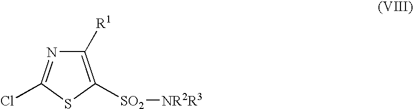

in which

R¹, R² and R³ have the abovementioned meaning,

and in a last step carrying out a reaction with amines of the general formula (IX)

H₂N—R^{4′} (IX)

in which

R^{4′} has the abovementioned meaning of R⁴ and is identical to or different from the latter, but is not hydrogen,

in inert solvents and in the presence of a base.

The compounds of the general formula (II)

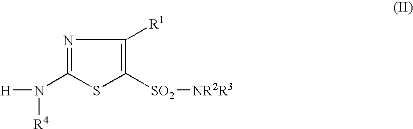

in which

R¹, R², R³ and R⁴have the meaning indicated in claim 1 are valuable intermediates and the present invention therefore also relates to them.

The reaction with chlorosulfonic acid/SO₂Cl takes place initially at room temperature and subsequently under the reflux temperature of the particular ether.

The reaction is generally carried out under atmospheric pressure. It is, however, also possible to carry out the process under elevated pressure or under reduced pressure (e.g. in a range from 0.5 to 5 bar).

Solvents suitable for the reaction with the amines of the general formula (V) are alcohols such as, for example, methanol, ethanol, propanol and isopropanol. Methanol is preferred.

The reaction with the amines of the general formula (V) takes place initially at room temperature and subsequently under the reflux temperature of the particular ether.

The reaction is generally carried out under atmospheric pressure. It is, however, also possible to carry out the process under elevated pressure or under reduced pressure (e.g. in a range from 0.5 to 5 bar).

The reaction with the compounds of the general formula (IX) takes place in ethers such as, for example, diethyl ether, dioxane, tetrahydrofuran or glycol dimethyl ether. Methanol is preferred.

Bases which can generally be employed are inorganic or organic bases. These preferably include organic amines (tri(C₁-C₆)alkylamines such as triethylamine), or heterocycles such as 1,4-diazabicyclo[2.2.2]octane (DABCO), 1,8-diazabicyclo[5.4.0]undec-7-ene (DBU), pyridine, diaminopyridine, methylpiperidine or morpholine. Triethylamine is preferred.

The base is generally employed in an amount of from 0.05 mol to 10 mol, preferably from 1 mol to 2 mol, based on 1 mol of the compound of the formula (VIII).

The compounds of the general formula (VI) are in some cases known or can be prepared by conventional methods [cf. Hantzsch, Chem. Ber. 1927, 60, 2544].

The compounds of the general formula (VII) and (VIII) are novel and can be prepared as described above.

Amines of the general formulae (V) and (IX) are known.

Compounds of the general formulae (III) are known or can be prepared by processes known from the literature.

Biphenylmethylcarboxylic acid or biphenylacetic acid derivatives of the formula (III) can be prepared in a manner known per se by transition metal-catalyzed, for example palladium-catalyzed, coupling reactions such as, for example, the Suzuki or Stille coupling. The pyridylphenylmethylcarboxylic acid derivatives of the formula (III) are known from the literature (see, for example, M. Artico et al. Eur. J. Med. Chem. (1992), 27, 219-228) or can be prepared by processes known per se. The following reaction schemes A, B, C and D illustrate by way of example the synthesis of biphenylacetic acid derivatives from the corresponding boronic acids, and the synthesis of pyridylphenylacetic acid derivatives from the corresponding stannyl compounds:

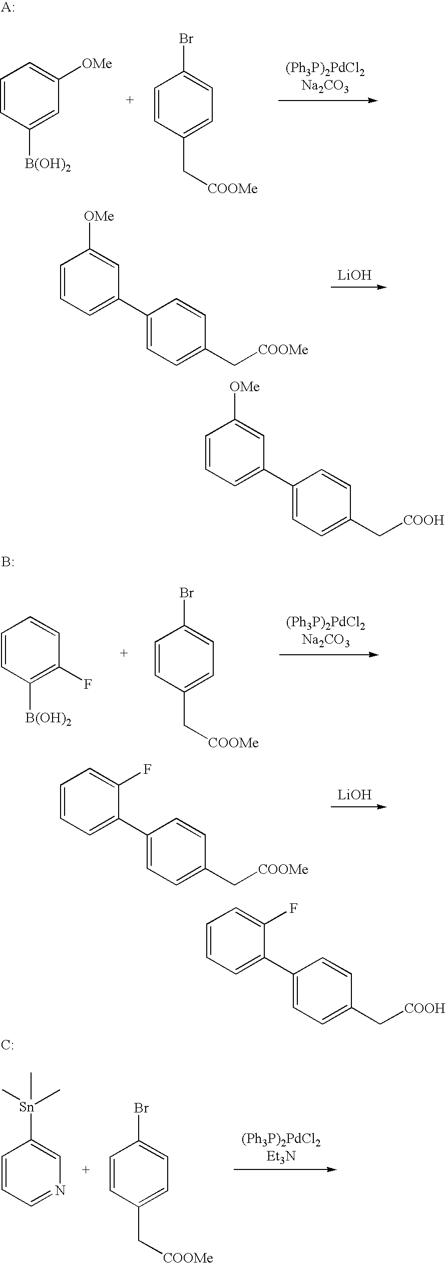

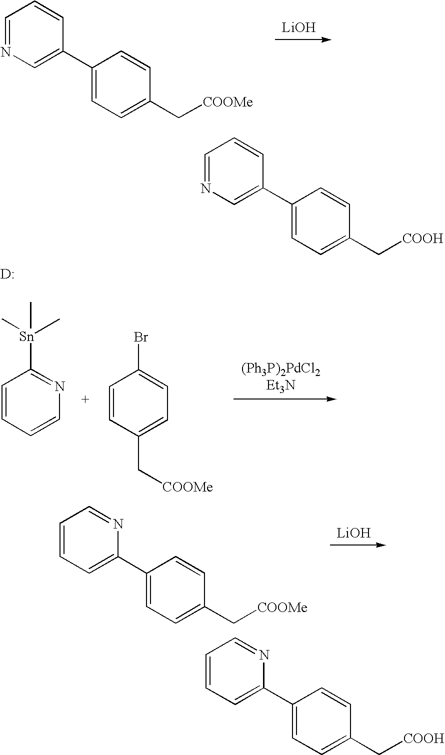

Compounds of the formula (III) in which R⁵ and R⁷ is, for example, fluorine can be prepared by the process shown in the following reaction scheme:

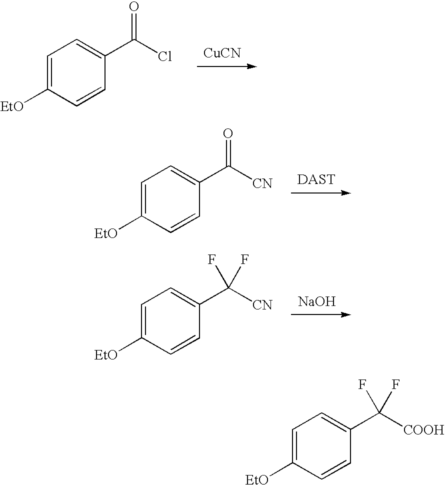

The fluorination with DAST (N,N-diethylaminosulfur trifluoride) in this case takes place as described in J. Fluor. Chem. 61, 1993, 117.

The invention further relates to the compounds of the formula (I) for the use as medicaments.

The invention further relates to a pharmaceutical composition which comprises a compound of the general formula (I) mixed with at least one pharmaceutically acceptable carrier or excipient.

The invention further relates to the use of a compound of the general formula (I) for producing a medicament, in particular a medicament for the treatment and/or prevention of viral infections, such as herpes viruses, especially herpes simplex viruses.

The compounds of the invention of the general formulae (I) show a surprising range of effects which could not have been predicted. They show an antiviral effect on representatives of the Herpes viridae group, in particular on herpes simplex viruses (HSV). They are thus [lacuna] for the treatment and prophylaxis of diseases caused by herpes viruses, especially diseases caused by herpes simplex viruses.

In vitro Activity

Viruses and Cells:

HSV (HSV-1 Walki, HSV-1F or HSV-2G) is replicated on Vero cells (ATCC CCL-81) under the following conditions: the cells are grown in M199 medium (5% fetal calf serum, 2 mM glutamine, 100 IU/ml penicillin, 100 μg/ml streptomycin) in cell culture bottles at 37° C. and 5% CO₂. The cells are split 1:4 on each of two occasions each week. For the infection, the medium is removed, and the cells are washed with Hank's solution, detached with 0.05% trypsin, 0.02% EDTA (Seromed L2143) and incubated at a density of 4×10⁵ cells per ml under the conditions mentioned above for 24 hours. The medium is then removed, and the virus solution is added at an m.o.i. of <0.05 in a volume of 2 ml per 175 cm² of surface area. After incubation under the conditions mentioned for one hour, the medium is made up to a volume of 50 ml per 175 cm² bottle. 3 days after the infection the cultures show clear signs of a cytopathic effect. The virus was released by freezing (−80° C.) and thawing (37° C.) twice. The cell debris is removed by centrifugation (300 g, 10 min, 4° C.) and the supernatant is frozen in aliquots at −80° C.

The virus titer is determined by a plaque assay. For this purpose, Vero cells are seeded in a density of 4×10⁵ cells per well in 24-well plates and, after incubation (37° C., 5% CO₂) for 24 hours, infected with dilutions of the virus stock of from 10⁻² to 10⁻¹² (100 μl inoculum). 1 hour after the infection, the medium is removed and the cells are covered with 1 ml of overlay medium (0.5% methylcellulose, 0.225 [lacuna] sodium bicarbonate, 2 mM glutamine, 100 IU/ml penicillin, 100 μg/ml streptomycin, 5% fetal calf serum in MEM Eagle medium with Earl's salt) and incubated for 3 days. The cells are subsequently fixed with 4% formalin for 1 hour, washed with water, stained with Giemsa (Merck) for 30 min and subsequently washed and dried. The virus titer is determined using a plaque viewer. The virus stocks used for the experiments have a titer of 1×10⁶/ml -1×10⁸/ml.

The anti-HSV effect is determined in a screening test system in 96-well microtiter plates with the assistance of various cell lines of neuronal, lymphoid and epithelial origin, such as, for example, Vero (African green monkey kidney cell line), MEF (murine embryonic fibroblasts), HELF (human embryonic fibroblasts), NT2 (human neuronal cell line) or Jurkat (human lymphoid T-cell line). The effect of the substances on the extent of the cytopathogenic effect is determined by comparison with the reference substance acyclovir sodium (Zovirax®), a clinically approved anti-herpes chemotherapeutic agent.

The substances dissolved (50 mM) in DMSO (dimethyl sulfoxide) are investigated on microtiter plates (for example 96-well MTP) in final concentrations of 250-0.5 μM (micromolar) in duplicate determinations (4 substances/plate). For potent substances, the dilutions are continued over several plates as far as 0.5 pM (picomolar). Toxic and cytostatic effects of the substance are also recorded. After the appropriate dilutions of the substance (1:2) on the microtiter plate, in medium a suspension of cells (1×10⁴ cells per well) such as, for example, Vero cells in M199 (medium 199) with 5% fetal calf serum, 2 mM glutamine and optionally 100 IU/ml penicillin and 100 μg/ml streptomycin or MEF cells in EMEM (Eagle's minimum essential medium) with 10% fetal calf serum, 2 mM glutamine and optionally 100 IU/ml penicillin and 100 μg/ml streptomycin, or HELF cells in EMEM with 10% fetal calf serum, 2 mM glutamine and optionally 100 IU/ml penicillin and 100 μg/ml streptomycin, or NT2 and Jurkat cells in DMEM (4.5 mg/l glucose plus pyridoxine) with 10% fetal calf serum 2 mM glutamine, 1 mM sodium pyruvate, nonessential amino acids and optionally 100 IU/ml penicillin and 100 μg/ml streptomycin is put in each well, and the cells in the relevant wells are infected with an appropriate amount of virus (HSV-1 F or HSV-2 G with an m.o.i (multiplicity of infection) of 0.0025 for HELF, Vero and MEF cells and with an m.o.i of 0.1 for NT2 and Jurkat cells). The plates are then incubated at 37° C. in a CO₂ incubator (5% CO₂) for several days. After this time, the cell lawn of, for example, Vero cells in the substance-free virus controls, starting from 25 infectious centres, is completely lysed or destroyed (100% CPE) by the cytopathogenic effect of the HSV viruses. The plates are initially evaluated optically using a microscope and then analyzed with a fluorescent dye. For this purpose, the cell culture supernatant is aspirated out of all the wells of the MTP and charged with 200 μl of PBS washing solution. The PBS is again aspirated out, and all the wells are charged with 200 μl of fluorescent dye solution (fluorescein diacetates, 10 μg/ml in PBS). After an incubation time of 30-90 min, the test plates are measured in a fluorimeter at an excitation wavelength of 485 nm and an emission wavelength of 538 nm.

IC₅₀ here means the half-maximum fluorescence intensity in relation to the uninfected cell control (100% value). The IC₅₀ may also be based on a suitable active ingredient control (see assay description: infected cells in the presence of a substance with anti-herpes effect of suitable concentration, such as, for example, Zovirax 20 μM). This active ingredient control reaches approximate fluorescence intensities of 85 to 95% in relation to the cell control.

Preference is given to N-[5-(aminosulfonyl)-1,3-thiazol-2-yl]acetamide derivatives of the invention whose IC₅₀ (HSV-1 F/Vero) in the in vitro screening test system described above is preferably less than 50 μM, more preferably less than 25 μM and very particularly preferably less than 10 μM.

The compounds according to the invention thus represent valuable active ingredients for the treatment and prophylaxis of disorders caused by herpes viruses, in particular herpes simplex viruses. Areas of indication which may be mentioned by way of example are:

1) Treatment and prophylaxis of herpes infections, in particular herpes simplex infections, in patients with pathological states such as herpes labialis, herpes genitalis, and HSV-related keratitis, encephalitis, pneumonia, hepatitis etc.

2) Treatment and prophylaxis of herpes infections, in particular herpes simplex infections, in immunosuppressed patients (for example AIDS patients, cancer patients, patients with genetically related immunodeficiency, transplant patients)

3) Treatment and prophylaxis of herpes infections, in particular herpes simplex infections, in neonates and infants

4) Treatment and prophylaxis of herpes infections, in particular herpes simplex infections, and patients positive for herpes, in particular for herpes simplex, to suppress recurrence (suppression therapy)

In vivo Effect

Animals:

6-week old female mice, strain BALB/cABom, are purchased from a commerical breeder (Bomholtgard Breeding and Research Centre Ltd.).

Infection:

The animals are anesthetized with diethyl ether (Merck) in a sealed glass vessel. 50 μl of a dilution of the virus stock (infectious dose 5×10⁴ pfu) are introduced into the nose of the anesthetized animals with an Eppendorf pipette. This infectious dose leads to death of 90-100% of the animals [lacuna] an average of between 5 and 8 days due to a generalized infection with prominent respiratory and central nervous symptoms.

Treatment and Evaluation:

6 hours after the infection, the animals are treated with doses of 0.1-100 mg/kg of body weight 3 times a day at 7.00 h, 14.00 h and 19 h over a period of 5 days. The substances are dissolved in DMSO and resuspended in Tylose/PBS (Hoechst) (final concentration 1.5% DMSO, 0.5% Tylose in PBS).

After the last administration, the animals are observed further and the times of death are recorded.

The novel active ingredients can be converted in a known manner into conventional formulations such as tablets, coated tablets, pills, granules, aerosols, syrups, emulsions, suspensions and solutions using inert, nontoxic, pharmaceutically suitable carriers and solvents. In these, the therapeutically active compound should in each case be present in a concentration of about 0.5 to 90% by weight of the complete mixture, that is to say in amounts which suffice to reach the dosage range indicated.

The formulations are produced, for example, by extending the active ingredients with solvents and/or carriers, where appropriate with use of emulsifiers and/or dispersants, it being possible for example in the case of use of water as diluent where appropriate to use organic solvents as auxiliary solvents.

Administration takes place in a conventional way, preferably orally, parenterally or topically, in particular perlingually or intravenously.

In the case of parenteral adminstration, solutions of the active substance using suitable liquid carrier materials can be employed.

It has generally proved advantageous in order to achieve effective results to administer amounts of about 0.001 to 20 mg/kg, preferably about 0.01 to 10 mg/kg of body weight on intravenous administration, and the dosage on oral administration is about 0.01 to 30 mg/kg, preferably 0.1 to 20 mg/kg of body weight.

It may, nevertheless, be necessary where appropriate to deviate from the amounts mentioned, in particular depending on the body weight and the nature of the administration route, on the individual behavior in response to the medicament, the nature of the formulation and the time or interval over which adminstration takes place. Thus, in some cases, it may be sufficient to make do with less than the aforementioned minimum amount, while in other cases the upper limit mentioned must be exceeded. In the case of administration of larger amounts it may be advisable to divide these into a plurality of individual doses over the day.

It may, where appropriate, be worthwhile to combine the compounds according to the invention with other active ingredients in particular antiviral active ingredients.

Starting Compounds

### EXAMPLE I

### 2-Chloro-4-methyl-1,3-thiazole-5-sulfonyl chloride

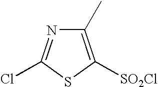

150 g (1.12 mol) of 2-chloro-4-methyl-1,3-thiazole are added dropwise to a solution of 331 g (2.81 mmol) of thionyl chloride in 653 g (5.61 mmol) of chlorosulfonic acid at room temperature. The solution is heated to reflux for 48 h. The mixture is then added to 3 l of ice-water and extracted with 4×400 ml of dichloromethane. The combined organic phases are washed with 2.5 l of water, dried over sodium sulfate and concentrated. Distillation of the crude product results in 233.7 g of product in the form of an oil. (boiling point 87-96° C., 0.7 mbar, GC 98.1%, yield 89.6%).

### EXAMPLE II

### Methyl [4-(2-pyridinyl)phenyl]acetate

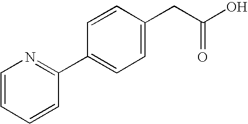

7.85 g (34.3 mmol) of methyl 4-bromophenolacetate are introduced under argon into 95 ml of toluene and, at room temperature, 7.97 g (61.7 mmol) of diisopropylethylamine, 9.50 g (37.7 mmol) of 2-trimethylstannylpyridine and 0.4 g (0.3 mmol) of tetrakis(triphenylphosphane)palladium (0) are added. The mixture is then heated under reflux for 18 h. After cooling, it is washed with 100 ml each of 1N hydrochloric acid and saturated sodium bicarbonate solution. The organic phase was discarded. The acidic and the basic aqueous phase were neutralized and extracted with 100 ml of dichloromethane in each case, and the combined organic phases were dried over magnesium sulfate and freed of solvent in vacuo. Example VII is obtained as a colorless oil after silica gel chromatography (toluene/ethyl acetate gradient 5:1-1:1).

Yield: 1.6g(19%) ¹H—NMR (400 MHz, d⁶-DMSO, δ/ppm): 3.64 (s, 3H), 3.76 (s, 2H), 7.33-7.40 (m, 1H), 7.39 (d, J=8.2 Hz; 2H), 7.86-7.90 (m, 1H), 7.96 (d, J=8.0 Hz; 1H), 8.05 (d, J=8.2 Hz; 2H), 8.67 (d, J=4.2 Hz, broad; 1H).

### EXAMPLE III

### [4-(2-Pyridinyl)phenyl]acetic acid

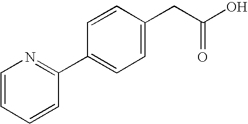

700 mg (3.11 mol) of methyl [4-(2-pyridinyl)phenyl]acetate are introduced into 5 ml of tetrahydrofuran and, at room temperature, 6.2 ml of a 1M potassium hydroxide solution in water are added. The mixture is then stirred at room temperature for 18 h, the solvent is subsequently substantially removed in vacuo, the residue is taken up in 10 ml of water, and the pH is adjusted to about 5 with 2N hydrochloric acid. Extraction of the aqueous phase twice with 10ml of dichloromethane each time afforded after drying of the combined organic phases over magnesium sulfate and removal of the solvent in vacuo the compound of example VIII in the form of a solid.

Yield: 300 mg (46%) ¹H—NMR (400 MHz, d⁶-DMSO, δ/ppm): 3.76 (s, 2H), 7.45-7.51 (m, 1H), 7.50 (d, J=8.3 Hz; 2H), 8.00 (td, J₁=7.7 Hz, J₂=1.9 Hz; 1H), 8.07 (d, J=7.9 Hz; 1H), 8.15 (d, J=8.3 Hz; 2H), 8.78 (d, J₁=4.0 Hz, J₂=0.9 Hz; 1H).

### EXAMPLE IV

### trans-2-Chloro-4-methyl-N-(2-phenylcyclopropyl)-1,3-thiazole-5-sulfonamide

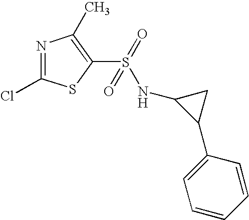

1.16 g (5.0 mmol) of 2-chloro-4-methyl-1,3-thiazole-5-sulfonyl chloride and 1.4 g (11 mmol) of diisopropylethylamine are dissolved in 10 ml of tetrahydrofuran and, at 0° C., a suspension of 0.93 g (5.5 mmol) of trans-2-phenylcyclopropylamine hydrochloride is added in portions. Stirring at room temperature overnight is followed by concentration, taking up in dichloromethane and extraction with water. Drying of the organic phase and concentration result in 1.0 g (HPLC purity: 88%) of yellowish oil which is not purified further but is reacted further.

The following is obtained in the same way:

### EXAMPLE V

trans-2-Chloro-N-fluorocyclopropyl-4-methyl-1,3-thiazole-5-sulfonamide

### EXAMPLE V

### 4-Methyl-trans-2-(methylamino)-N-(2-phenylcyclopropyl)-1,3-thiazole-5-sulfonamide

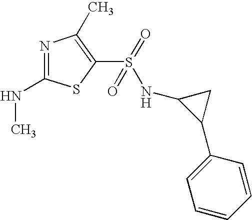

0.69 ml of a 40% solution of methylamine in water is added to a solution of 0.95 g (2.9 mmol) of trans-2-chloro-4-methyl-N-(2-phenylcyclopropyl)-1,3-thiazole-5-sulfonamide in 10 ml of acetonitrile. The solution is heated at 50° C. overnight. After cooling to room temperature, the precipitated solid are filtered off and further purified by chromatography (RP18, acetonitrile/water gradient 30/70→95/5). MS (ESIpos): m/z=324 [M+H]⁺

### PREPARATIONS EXAMPLES

### Example 1

### N-Methyl-N-(4-methyl-5-{[trans-(2-phenylcyclopropyl)amino]sulfonyl }-1,3-thiazol-2-yl)-2-[4-(2-pyridinyl)phenyl]acetamide

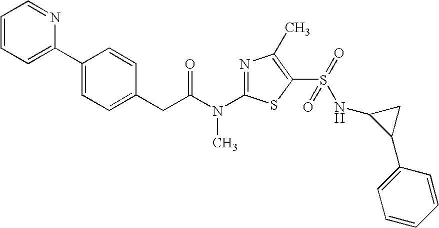

0.32 g (1.5 mmol) of 4-pyridophenylacetic acid (Eur. J. Med. Chem. Chim. Ther.; 27; 3; 1992; 219-228) and 0.32 g (1.6 mmol) of N-(3-dimethylaminopropyl)-N′-ethylcarbodiimide are introduced into 4 ml of acetonitrile at room temperature. 0.53 g (1.6 mmol) of 4-methyl-trans-2-(methylamino)-N-(2-phenylcyclopropyl)-1,3-thiazole-5-sulfonamide and 0.22 g (1.6mmol) of 1-hydroxy-1H-benzotriazole are added, and the mixture is stirred at room temperature for 4 days. This is followed by removal of the solvent by distillation, partition between dichloromethane and water and evaporation of the organic solvent. The resulting oil is further purified by reversed phase chromatography (RP18, acetonitrile/water gradient 30/70→95/5); yield 0.39 g (50%) of colorless oil.

MS (ESIpos): m/z=519 [M+H]⁺

¹H—NMR (DMSO-d₆, 400 MHz): δ=1.15 (m, 2H), 1.85 (m, 1H), 2.35 (s, 3H), 2.4 (m, 1H), 3.75 (s, 3H), 4.25 (s, 2H), 6.9 (dd, 2H), 7.1-7.25 (m, 3H), 7.35-7.45 (m, 3H), 7.85 (m, 1H), 7.95 (dd, 1H), 8.1 (dd, 2H), 8.35 (d, 1H), 8.7 (d, 1H).

1. A compound of the formula (I): 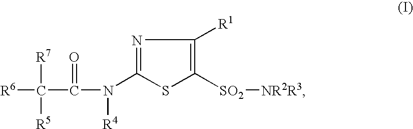
in which
R¹ is hydrogen, halogen, (C₁-C₆)-alkyl, (C₁-C₆)-alkoxy, amino-(C₁-C₆)-alkyl or halo-(C₁-C₆)-alkyl,
R² is hydrogen, (C₁-C₆)-alkoxy, (C₃-C₈)-cycloalkyl or biphenylaminocarbonyl, or
is (C₁-C₆)-alkyl which is optionally substituted by 1 to 3 substituents which are selected from the group consisting of (C₃-C₆)-cycloalkyl, (C₁-C₆)-alkoxy, halogen, hydroxyl, amino, tri-(C₁-C₆)-alkylsilyloxy, radicals of the formula 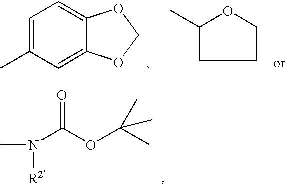
in which R^{2′} is hydrogen or (C₁-C₄)-alkyl,
a 5- to 6-membered aromatic heterocycle having up to 3 heteroatoms from the series S, N and/or O, where a nitrogen-containing heterocycle may also be bonded via the nitrogen atom,
a 3- to 8-membered saturated or unsaturated, nonaromatic, heterocycle which is optionally bonded via a nitrogen atom and has up to 3 heteroatoms from the series S, N and/or O, and (C₆-C₁₀)-aryl which may in turn be substituted by hydroxyl or (C₁-C₆)-alkoxy, or
is a group of the formula 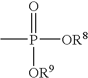
in which R⁸ and R⁹ are identical to or different from one another and are hydrogen and (C₁-C₄)-alkyl, or
are a group of the formula 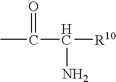
in which R¹⁰ is the side group of a naturally occurring α-amino acid, or
is a group of the formula 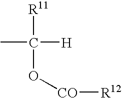
in which R¹¹ is (C₁-C₄)-alkyl, and R¹² is hydrogen, (C₁-C₄)-alkyl or a group of the formula 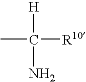
in which R^{10′}is the side group of a naturally occurring α-amino acid,
or in which
R² is (C₃-C₈)-cycloalkyl,
which is optionally substituted by 1 to 3 substituents selected from the group consisting of (C₁-C₆)-alkyl, (C₃-C₆)-cycloalkyl, (C₁-C₆)-alkoxy, trifluoromethyl, trifluoromethoxy, halogen, hydroxyl, amino and nitro,
R³ is (C₃-C₈)-cycloalkyl,
which is substituted by 1 to 3 substituents selected from the group consisting of (C₁-C₆)-alkyl, (C₃-C₆)-cycloalkyl, (C₁-C₆)-alkoxy, (C₁-C₆)-alkylcarbonylamino, carboxyl, (C₁-C₆)-alkoxycarbonyl, mono- or di(C₁-C₆)-alkylamino, mono- or di(C₁-C₆)-alkyl-aminocarbonyl, (C₁-C₆)-alkylsulfoxy, (C₁-C₆)-alkylsulfonyl, (C₆-C₁₀)-arylsulfoxy, (C₆-C₁₀)-arylsulfonyl, (C₁-C₆)-alkylamino-sulfonyl, (C₁-C₆)-dialkylaminosulfonyl, hydroxymethyl, trifluoromethyl, trifluoromethoxy, halogen, hydroxyl, amino, nitro and (C₆-C₁₀)-aryl,
where (C₆-C₁₀)-aryl may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-lkyl, halo-(C₁-C₆)-lkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl,
R⁴ is hydrogen, (C₁-C₆)-acyl, (C₂-C₆)-alkenyl, (C₃-C₈)-cycloalkyl, or
R⁴ is (C₁-C₆)-alkyl which may be optionally substituted by 1 to 3 substituents which are selected from the group consisting of halogen, hydroxyl, (C₃-C₈)-cycloalkyl, (C₁-C₆)-acyl, (C₁-C₆)-alkoxy, carboxyl, 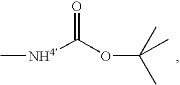
in which R^{4′} is hydrogen,
—(OCH₂CH₂)ₙOCH₂CH₃ in which n is 0 or 1, phenoxy, (C₆-C₁₀)-aryl and —NR¹³R¹⁴,
in which R¹³ and R¹⁴ are identical or different and are hydrogen, (C₁-C₆)-acyl, (C₁-C₆)-alkyl, carbamoyl, mono- or di(C₁-C₆)-alkylamino(C₁-C₆)-alkyl, mono- or di(C₁-C₆)-alkylaminocarbonyl, (C₆-C₁₀)-aryl or (C₁-C₆)-alkoxycarbonyl or
R¹³ and R¹⁴ form together with the nitrogen atom a 5- to 6-membered saturated heterocycle which may optionally comprise a further heteroatom from the series S or O or a radical of the formula —NR¹⁵, and may be substituted by oxo,
in which
R^{1 5} is hydrogen or (C₁-C₄)-alkyl, or
R⁴ is (C₁-C₆)-alkyl which is substituted by a 5- to 6-membered aromatic, optionally benzo-fused heterocycle having up to 3 heteroatoms from the series S, N and/or O, where a nitrogen-containing heterocycle may also be bonded via the nitrogen atom, or is substituted by
radicals of the formulae 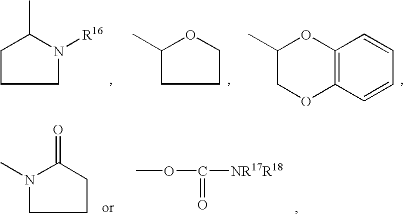
in which
R¹⁶ is hydrogen or (C₁-C₆)-alkyl,
R¹⁷ and R¹⁸ are identical or different and are hydrogen, (C₁-C₆)-alkyl or (C₆-C₁₀)-aryl, where aforementioned (C₁-C₆)-alkyl and (C₆-C₁₀)-aryl may optionally be substituted by 1 to 3 substituents selected from the group consisting of hydroxyl, (C₁-C₆)-alkoxy and halogen,
R⁵ is hydrogen, (C₁-C₆)-alkyl, halogen, amino, mono- or di(C₁-C₆)-alkylamino or is (C₁-C₆)-alkanoylamino,
R⁶ is phenyl which may optionally be substituted by one to three substituents selected from the group consisting of
halogen,
(C₆-C₁₀)-aryl which may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-lkyl, halo-(C₁-C₆)-alkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl, tri-(C₁-C₆)-alkylsilyloxy, a 3- to 8-membered, saturated or unsaturated, nonaromatic, mono- or bicyclic heterocycle which is optionally bonded via a nitrogen atom and has up to 3 heteroatoms from the series S, N and/or O, and/or cyano,
(C₁-C₆)-alkoxy,
(C₁-C₆)-alkoxycarbonyl,
(C₁-C₆)-alkylthio,
hydroxyl,
carboxyl,
partially fluorinated (C₁-C₆)-alkoxy having up to 6 fluorine atoms,
(C₁-C₆)-alkyl which is optionally substituted by a radical of the formula 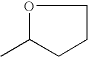
a 5- to 6-membered aromatic heterocycle which is optionally bonded via a nitrogen atom and has up to 3 heteroatoms from the series S, N and/or O and which may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-alkyl, halo-(C₁-C₆)-alkoxy, amino, (C₁-C₆)-alkylthio, hydroxyl, carboxyl, carbamoyl, aminocarbonyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl, a 3- to 8-membered saturated or unsaturated, nonaromatic, mono- or bicyclic heterocycle which is optionally bonded via a nitrogen atom and has up to 3 heteroatoms from the series S, N and/or O, and/or cyano,
a 3- to 8-membered saturated or unsaturated, nonaromatic, mono- or bicyclic heterocycle which is optionally bonded via a nitrogen atom and has up to 3 heteroatoms from the series S, N and/or O, and which may optionally be substituted by 1 to 3 substituents selected from oxo, halogen, hydroxyl, (C₁-C₆)-alkoxycarbonyl, (C₁-C₆)-alkoxycarbonylamino, (C₁-C₆)-alkyl, halo-(C₁-C₆)-alkyl and hydroxy-(C₁-C₆)-alkyl,
(C₂-C₆)-alkenyl
and groups of the formulae
OR¹⁹,
—NR²⁰R²¹ or —CO—NR²²R²³,
carbazole, dibenzofuran or dibenzothiophene,
xanthene or 9,10-dihydroacridine,
in which R¹⁹ is phenyl which in turn is optionally substituted by a group of the formula —NR²⁴R²⁵,
in which
R²⁴ and R²⁵ are identical or different and are hydrogen, (C₁-C₆)-alkyl or (C₁-C₆)-acyl,
or
R¹⁹ is (C₁-C₆)-alkyl which is optionally substituted once to three times by hydroxyl and/or halogen,
R²⁰ and R²¹ are identical or different and are hydrogen, carbamoyl, mono- or di-(C₁-C₆)-alkylaminocarbonyl, phenyl, (C₁-C₆)-acyl or (C₁-C₆)-alkyl,
where aforementioned (C₁-C₆)-alkyl is optionally substituted by (C₁-C₆)-alkoxy, (C₁-C₆)-acyl, by phenyl or by a 5- to 6-membered aromatic heterocycle having up to 3 heteroatoms from the series S, N and/or O,
where aforementioned phenyl and aforementioned aromatic heterocycle are optionally substituted once to three times, identically or differently, by halogen 5 and/or hydroxyl, and
R²² and R²³ are identical or different and are hydrogen or (C₁-C₆)-alkyl,
and R⁷ may have the meaning of R⁵ and may be identical to or different from the latter,
and the salts thereof.

2. A compound of the formula (I) as claimed in claim 1, in which
in which
R¹ is hydrogen, halogen, (C₁-C₆)-alkyl, (C₁-C₆)-alkoxy, amino-(C₁-C₆)-alkyl or halo-(C₁-C₆)-alkyl,
R² is hydrogen, (C₃-C₈)-cycloalkyl or biphenylaminocarbonyl, or
is (C₁-C₆)-alkyl which is optionally substituted by 1 to 3 substituents which are selected from the group consisting of (C₃-C₆)-cycloalkyl, (C₁-C₆)-alkoxy, halogen, hydroxyl, amino, tri-(C₁-C₆)-alkylsilyoxy, radicals of the formula 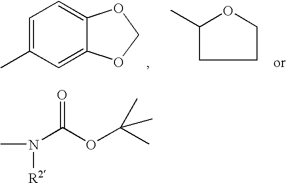
in which R^{2′} is hydrogen or (C₁-C₄)-alkyl,
a 5- to 6-membered aromatic heterocycle having up to 3 heteroatoms from the series S, N and/or O, where a nitrogen-containing heterocycle may also be bonded via the nitrogen atom,
a 3- to 8-membered saturated or unsaturated, nonaromatic, heterocycle which is optionally bonded via a nitrogen atom and has up to 3 heteroatoms from the series S, N and/or O, and
(C₆-C₁₀)-aryl which may in turn be substituted by hydroxyl or (C₁-C₆)-alkoxy, or
is a group of the formula 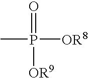
in which R⁸ and R⁹ are identical to or different from one another and are hydrogen and (C₁-C₄)-alkyl, or
are a group of the formula 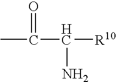
in which R¹⁰ is the side group of a naturally occurring α-amino acid, or
is a group of the formula 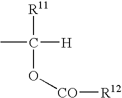
in which R¹¹ is (C₁-C₄)-alkyl, and R¹² is hydrogen, (C₁-C₄)-alkyl or is a group of the formula 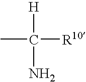
in which R^{10′} is the side group of a naturally occurring α-amino acid,
or in which
R² is (C₃-C₈)-cycloalkyl
which is optionally substituted by 1 to 3 substituents selected from the group consisting of (C₁-C₆)-alkyl, (C₃-C₆)-cycloalkyl, (C₁-C₆)-alkoxy, trifluoromethyl, trifluoromethoxy, halogen, hydroxyl, amino and nitro,
R³ is (C₃-C₈)-cycloalkyl
which is substituted by 1 to 3 substituents selected from the group consisting of (C₁-C₆)-alkyl, (C₃-C₆)-cycloalkyl, (C₁-C₆)-alkoxy, (C₁-C₆)-alkylcarbonylamino, carboxyl, (C₁-C₆)-alkoxycarbonyl, mono- or di-(C₁-C₆)-alkylamino, mono- or di-(C₁-C₆)-alkylaminocarbonyl, (C₁-C₆)-alkylsulfoxy, (C₁-C₆)-alkylsulfonyl, (C₆-C₁₀)-arylsulfoxy, (C₆-C₁₀)-arylsulfonyl, (C₁-C₆)-alkylaminosulfonyl, (C₁-C₆)-dialkylaminosulfonyl, hydroxymethyl, trifluoromethyl, trifluoromethoxy, halogen, hydroxyl, amino, nitro and (C₆-C₁₀)-aryl,
where (C₆-C₁₀)-aryl may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-lkyl, halo-(C₁-C₆)-lkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl,
R⁴ is hydrogen, (C₁-C₆)-acyl, (C₂-C₆)-alkenyl, (C₃-C₈)-cycloalkyl, or R⁴ is (C₁-C₆)-alkyl which may optionally be substituted by 1 to 3 substituents which are selected from the group consisting of halogen, hydroxyl, (C₃-C₈)-cycloalkyl, (C₁-C₆)-acyl, (C₁-C₆)-alkoxy, carboxyl, 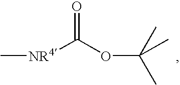
in which R^{4′} is hydrogen, —(OCH₂CH₂)ₙOCH₂CH₃ in which n is 0 or 1, phenoxy, (C₆-C₁₀)-aryl and —NR³R¹⁴,
in which R¹³ and R¹⁴ are identical or different and are hydrogen, (C₁-C₆)-acyl, (C₁-C₆)-alkyl, carbamoyl, mono- or di(C₁-C₆)-alkylamino(C₁-C₆)-alkyl, mono- or di(C₁-C₆)-alkylaminocarbonyl, (C₆-C₁₀)-aryl or (C₁-C₆)-alkoxycarbonyl, or
R¹³ and R¹⁴ form together with the nitrogen atom a 5- to 6-membered saturated heterocycle which may optionally comprise a further heteroatom from the series S or O or a radical of the formula —NR¹⁵, and may be substituted by oxo,
in which
R¹⁵ is hydrogen or (C₁-C₄)-alkyl, or
R⁴ is (C₁-C₆)-alkyl which is substituted by a 5- to 6-membered aromatic, optionally benzo-fused heterocycle having up to 3 heteroatoms from the series S, N and/or O, where a nitrogen-containing heterocycle may also be bonded via the nitrogen atom, or is substituted by
radicals of the formulae 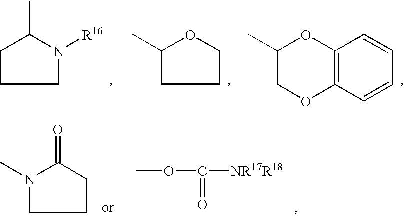
in which
R¹⁶ is hydrogen or (C₁-C₆)-alkyl,
R¹⁷ and R¹⁸ are identical or different and are hydrogen, (C₁-C₆)-alkyl or (C₆-C₁₀)-aryl, where aforementioned (C₁-C₆)-alkyl and (C₆-C₁₀)-aryl may optionally be substituted by 1 to 3 substituents selected from the group consisting of hydroxyl, (C₁-C₆)-alkoxy and halogen,
R⁵ is hydrogen, (C₁-C₆)-alkyl, halogen, amino, mono- or di(C₁-C₆)-alkylamino or is (C₁-C₆)-alkanoylamino,
R⁶ is phenyl which may optionally be substituted by one to three substituents selected from the group consisting of
halogen,
(C₆-C₁₀)-aryl which may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-lkyl, halo-(C₁-C₆)-alkoxy, amino, (C₁-C₆)-alkylthio, hydroxyl, carboxyl, carbamoyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl, tri-(C₁-C₆)-alkylsilyloxy, a 3- to 8-membered, saturated or unsaturated, nonaromatic, mono- or bicyclic heterocycle which is optionally bonded via a nitrogen atom and has up to 3 heteroatoms from the series S, N and/or O, and/or cyano,
(C₁-C₆)-alkoxy,
(C₁-C₆)-alkoxycarbonyl,
(C₁-C₆)-alkylthio,
hydroxyl,
carboxyl,
partially fluorinated (C₁-C₆)-alkoxy having up to 6 fluorine atoms,
(C₁-C₆)-alkyl which is optionally substituted by a radical of the formula 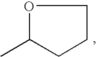
a 5- to 6-membered aromatic heterocycle which is optionally bonded via a nitrogen atom and carries up to 3 heteroatoms from the series S, N and/or O and which may optionally be substituted by 1 to 3 substituents selected from (C₁-C₆)-alkanoyl, (C₁-C₆)-alkoxy, (C₁-C₆)-alkyl, halogen, (C₁-C₆)-alkoxycarbonyl, nitro, halo-(C₁-C₆)-alkyl, halo-(C₁-C₆)-alkoxy, amino, (C₁-C₆)-alkylthio, hydroxyl, carboxyl, carbamoyl, aminocarbonyl, mono- or di-(C₁-C₆)-alkylaminocarbonyl, mono- or di-(C₁-C₆)-alkanoylamino, (C₁-C₆)-alkoxycarbonylamino, (C₁-C₆)-alkylsulfoxy, (C₁-C₆)-alkylsulfonyl, a 3- to 8-membered saturated or unsaturated, nonaromatic, mono- or bicyclic heterocycle which is optionally bonded via a nitrogen atom and has up to 3 heteroatoms from the series S, N and/or O, and/or cyano,
a 3- to 8-membered saturated or unsaturated, nonaromatic, mono- or bicyclic heterocycle which is optionally bonded via a nitrogen atom and has up to 3 heteroatoms from the series S, N and/or O, and which may optionally be substituted by 1 to 3 substituents selected from oxo, halogen, hydroxyl, (C₁-C₆)-alkoxycarbonyl, (C₁-C₆)-alkoxycarbonylamino, (C₁-C₆)-alkyl, halo-(C₁-C₆)-alkyl and hydroxy-(C₁-C₆)-alkyl, (C₂-C₆)-alkenyl,
and groups of the formulae
OR¹⁹,
—NR²⁰R²¹ or —CO—NR²²R²³,
carbazole, dibenzofuran or dibenzothiophene,
xanthene or 9,10-dihydroacridine,
in which R¹⁹ is phenyl which in turn is optionally substituted by a group of the formula —NR²⁴R²⁵,
in which
R²⁴ and R²⁵ are identical or different and are hydrogen, (C₁-C₆)-alkyl or (C₁-C₆)-acyl,
or
R¹⁹ is (C₁-C₆)-alkyl which is optionally substituted once to three times by hydroxyl and/or halogen,
R²⁰ and R²¹ are identical or different and are hydrogen, carbamoyl, mono- or di-(C₁-C₆)-alkylaminocarbonyl, phenyl, (C₁-C₆)-acyl or (C₁-C₆)-alkyl,
where aforementioned (C₁-C₆)-alkyl is optionally substituted by (C₁-C₆)-alkoxy, (C₁-C₆)-acyl, by phenyl or by a 5- to 6-membered aromatic heterocycle having up to 3 heteroatoms from the series S, N and/or O,
where aforementioned phenyl and aforementioned aromatic heterocycle are optionally substituted once to three times, identically or differently, by halogen and/or hydroxyl, and
R²² and R²³ are identical or different and are hydrogen or (C₁-C₆)-alkyl,
and R⁷ may have the meaning of R⁵ and may be identical to or different from the latter,
and the salts thereof.

3. A compound of the formula (I) as claimed in claim 1, in which R¹ is hydrogen or (C₁-C₆)-alkyl.

4. A compound of the formula (I) as claimed in claim 1, in which R² is hydrogen or (C₁-C₆)-alkyl.

5. A compound of the formula (I) as claimed in claim 1, in which R³ is cyclopropyl which is substituted by 1 to 2 substituents selected independently of one another from the group consisting of (C₁-C₃)-alkyl and halogen.

6. A compound of the formula (I) as claimed in claim 1, in which R⁴ is hydrogen or (C₁-C₆)-alkyl.

7. A compound of the formula (I) as claimed in claim 1, in which R⁵ is hydrogen.

8. A process for preparing the compounds of the general formula (I) as claimed in claim 1, characterized in that
compounds of the general formula (II) 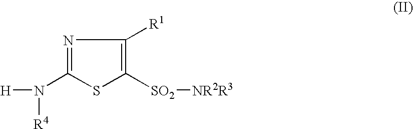
in which
R¹, R^{2,} R³ and R⁴ have the meaning stated in claim 1,
are reacted with compounds of the general formula (III) 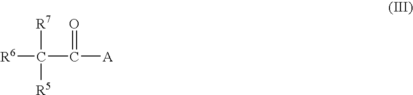
in which
A is a leaving group, and R⁵, R⁶ and R⁷ have the meaning stated in claim 1, to give compounds of the formula (I).

9. A compound of the formula (I) as claimed in claim 1 for controlling diseases.

10. A medicament comprising a compound of the formula (I) as claimed in claim 1 in combination with at least one pharmaceutically suitable, pharmaceutically acceptable carrier or excipient.

11. The use of compounds of the general formula (I) as claimed in claim 1 for production as medicament for the treatment of viral diseases.

12. A medicament as claimed in claim 11 for the treatment of viral diseases.

13. A method for controlling viral diseases in humans and animals by administration of an antivirally effective amount of at least one compound as claimed in claim 1.

14. A compound of the formula (II) 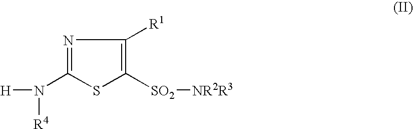
in which
R¹, R², R³ and R⁴ have the meaning stated in claim 1.
